(19) United States
(12) Patent Application Publication
(10) Pub. No.: US 2003/0105517 A1
(43) Pub. Date: Jun. 5, 2003

(54) Non-foreshortening stent

(76) Inventors: Geoffrey Hamilton White, Sydney (AU); Russell J. Redmond, Goleta, CA (US)

Correspondence Address: Raymond Sun, 12420 Woodhall Way, Tustin, CA, 92782, US

(21) Appl. No.: 10/010,612

(22) Filed: Dec. 5, 2001

(51) Int. Cl. A61F 2/06

(52) U.S. Cl. 623/1.17; 623/1.16

(57) ABSTRACT

A stent has a plurality of annular elements, each annular element having a compressed state and an expanded state, with each annular element having a longitudinal dimension which is smaller in the expanded state than in the compressed state. The stent also has at least one connecting member connecting adjacent annular elements, the connecting member having a longitudinal dimension which is larger in the expanded state than in the compressed state.

### BACKGROUND OF THE INVENTION

1. Field of the Invention

The present invention relates to an intraluminal prosthesis for implantation into a mammalian vessel, and in particular, to an intraluminal stent that is delivered in a compressed state to a specific location inside the lumen of a mammalian vessel and then deployed to an expanded state to support the vessel. The intraluminal stent is provided with a structural configuration that maintains the prosthesis at substantially the same length in both the compressed and expanded states.

2. Description of the Prior Art

Intraluminal prosthesis, such as stents, are commonly used in the repair of aneurysms, as liners for vessels, or to provide mechanical support to prevent the collapse of stenosed or occluded vessels. These stents are typically delivered in a compressed state to a specific location inside the lumen of a vessel or other tubular structures, and then deployed at that location of the lumen to an expanded state. The stent has a diameter in its expanded state which is several times larger than the diameter of the stent in its compressed state. These stents are also frequently deployed in the treatment of atherosclerotic stenosis in blood vessels, especially after percutaneous transluminal coronary angioplasty (PTCA) procedures, to improve the results of the procedure and to reduce the likelihood of restenosis.

The positioning of a stent at the desired location in the lumen of a body vessel is a critical factor that affects the performance of the stent and the success of the medical procedure. Since the region in a lumen at which the stent is to be deployed is usually very difficult for a physician to access, it is essential that the stent's deployed diameter and length be known before the physician can accurately position a stent with the correct size at the precise location. For example, since the diameter and the length of the diseased or damaged segment or region of the body vessel can vary for different body vessels, disease states, and deployment purposes, it is important that a stent having the precise diameter and length be delivered to this region for deployment.

Careful sizing of this region of the lumen of the body vessel may pose a difficult challenge for many physicians who know the exact dimensions of the body vessel at this region, but are not certain about the stent's deployed diameter and length. This is due to a foreshortening effect which is experienced by many stents when they are expanded from their compressed state to their expanded state.

This foreshortening effect is illustrated in FIGS. 1A, 1B, 2A and 2B, which illustrate portions 20 of a stent having a mesh-like pattern made up of V-shaped struts or legs 22 and 24 connected at their apices 26. Two pairs of these V-shaped struts 22, 24 are illustrated in this portion 20 of the stent. Each of these struts 22 and 24 has a length h. FIG. 1B illustrates the portion 20 of the stent in a fully compressed state, in which the length h has a longitudinal or horizontal component I₂ (see FIG. 2B), and FIG. 1A illustrates the same portion 20 of the stent in a fully expanded state, in which the length h has a longitudinal or horizontal component I₁ (see FIG. 2A). As illustrated by the imaginary lines 28 and 30 in FIGS. 1A and 1B, and in FIGS. 2A and 2B, I₁ is shorter than I₂ because the angle 0 which the strut 22 assumes with respect to the horizontal axis is greater when in the expanded state, so the length of the expanded portion 20 is shorter than the length of the compressed portion 20 by a length of 2d. This foreshortening is caused by the shortening of the longitudinal component I of the struts 22 and 24 as the stent is expanded from the compressed state to the expanded state.

This foreshortening effect is troublesome because it is not easy to determine the exact dimension of this foreshortened length 2d. The physician must make this calculation based on the material of the stent, the body vessel being treated, and the expected diameter of the stent when properly deployed in the lumen of the body vessel. For example, the foreshortened length 2d will vary when the same stent is deployed in vessels having different diameters at the region of deployment.

In addition, there are certain body vessels that experience a change in vessel lumen diameter, anatomy or disease state along their lengths. Stents to be deployed at such vessels will need to be capable of addressing or adapting to these changes.

An example of such a body vessel are the carotid arteries. Blood is delivered from the heart to the head via the common carotid arteries. These arteries are approximately 8-10 mm in lumen diameter as they make their way along the neck up to a position just below and behind the ear. At this point, the common carotid artery branches into a 6-8 mm lumen diameter internal carotid artery, which feeds blood to the brain, and a 6-8 mm lumen diameter external carotid artery, which supplies blood to the face and scalp. Atherosclerotic lesions of the carotid artery tend to occur around this bifurcation of the common carotid artery into the internal and external carotid arteries, so stents often need to be deployed at this bifurcation.

Another example are the iliac arteries, which have a lumen diameter of about 8-10 mm at the common iliac artery but which decrease to a lumen diameter of about 6-7 mm at the external iliac artery. The common iliac arteries experience more localized stenosis or occlusive lesion which are quite often calcific and usually require a shorter stent with greater radial strength or rigidity. More diffused atherosclerotic disease of the iliac system will commonly involve both the common and external iliac arteries, and necessitate a longer stent having increased flexibility that is suitable for deployment in the tortuous angulation experienced by the iliac system.

The femoropopliteal system similarly experiences localized and diffused stenotic lesions. In addition, the flexibility of a stent is important where deployed at locations of vessels that are affected by movements of joints, such as the hip joint or the knee joint.

The renal arteries provide yet another useful example. The initial 1 cm or so at the orifice of a renal artery is often quite firmly narrowed due to atheroma and calcification, and is relatively straight, while the remainder of the length of the renal artery is relatively curved. As a result, a stent intended for implantation at the renal arteries should be relatively rigid for its first 1.5 cm or so, and then become more flexible and compliant.

Thus, there remains a need for an intraluminal prosthesis that maintains a consistent length in both its fully compressed and fully expanded states, and in all states between its fully compressed and fully expanded states. There also remains a need for a stent which can accomodate body vessels having varying lumen diameters, different anatomies, and different disease states.

### SUMMARY OF THE DISCLOSURE

In order to accomplish the objects of the present invention, there is provided a stent having a plurality of annular elements, each annular element having a compressed state and an expanded state, with each annular element having a longitudinal dimension which is smaller in the expanded state than in the compressed state. The stent also has at least one connecting member connecting adjacent annular elements, the connecting member having a longitudinal dimension which is larger in the expanded state than in the compressed state. In one embodiment, the connecting member is straight when the annular elements are in the compressed state and in the expanded state. In another embodiment, the connecting member is straight when the annular elements are in the expanded state, and the connecting member is arcuate when the annular elements are in the compressed state.

### BRIEF DESCRIPTION OF THE DRAWINGS

FIG. 1A is a side elevational view of a portion of a prior art stent in its expanded state;

FIG. 1B is a side elevational view of the portion of FIG. 1A in its compressed state;

FIG. 2A illustrates the longitudinal component of a strut of the stent of FIGS. 1A and 1B when the stent is in its expanded state;

FIG. 2B illustrates the longitudinal component of a strut of the stent of FIGS. 1A and 1B when the stent is in its compressed state;

FIG. 3 is a side elevational view of a portion of a stent according to one embodiment of the present invention shown in a compressed state;

FIG. 4 is a side elevational view of the portion of FIG. 3 in its expanded state;

FIG. 5 is a side elevational view of the portion of FIG. 4 in its expanded state showing the removal of certain struts and connecting members;

FIG. 6 is a side elevational view of a portion of a stent according to another embodiment of the present invention shown in a compressed state;

FIG. 7 is a side elevational view of the portion of FIG. 6 in its expanded state;

FIG. 8 is a side elevational view of a portion of a stent according to yet another embodiment of the present invention shown in a compressed state;

FIG. 9 is a side elevational view of the portion of FIG. 8 in its expanded state;

FIG. 10 is a side elevational view of a portion of a stent according to yet another embodiment of the present invention shown in a compressed state;

FIG. 11 is a side elevational view of the portion of FIG. 10 in its expanded state;

FIG. 12 is a side elevational view of a portion of a stent according to yet another embodiment of the present invention shown in a compressed state;

FIG. 13 is a side elevational view of the portion of FIG. 12 in its expanded state;

FIG. 14 is a side elevational view of a portion of a stent according to yet another embodiment of the present invention shown in a compressed state;

FIG. 15 is a side elevational view of the portion of FIG. 14 in its expanded state;

FIG. 16 is a side elevational view of a portion of a stent according to yet another embodiment of the present invention shown in a compressed state; and

FIG. 17 is a side elevational view of the portion of FIG. 16 in its expanded state.

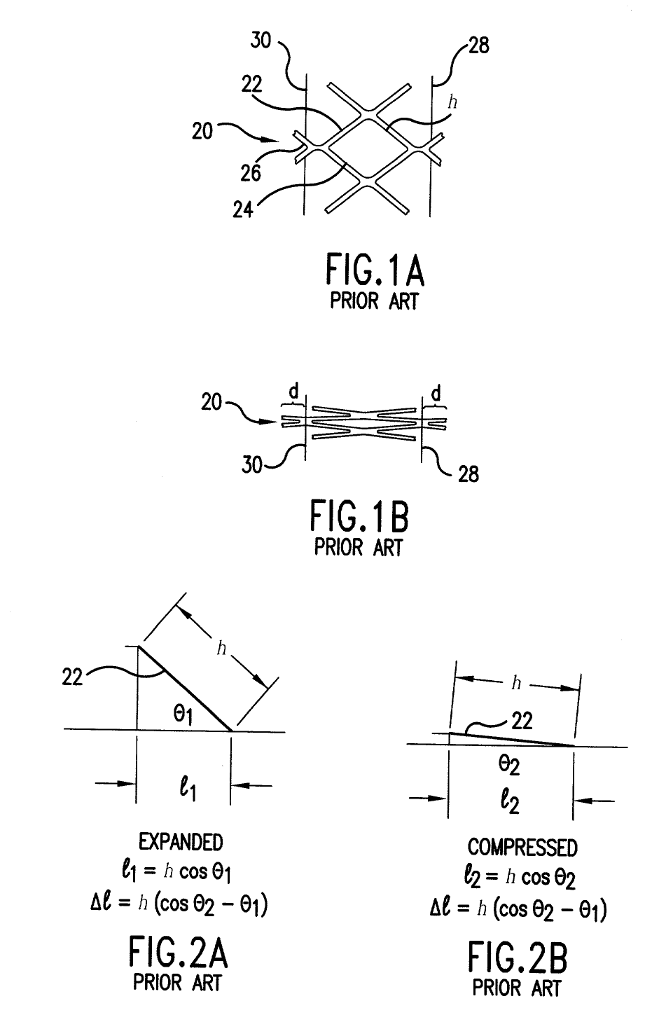

### DETAILED DESCRIPTION OF THE PREFERRED EMBODIMENTS

The following detailed description is of the best presently contemplated modes of carrying out the invention. This description is not to be taken in a limiting sense, but is made merely for the purpose of illustrating general principles of embodiments of the invention. The scope of the invention is best defined by the appended claims.

The intraluminal prosthesis according to the present invention is a stent, although the principles of the present invention are also applicable to other prosthesis such as liners and filters. The stent is delivered to a desired location in the lumen of a body vessel in a compressed state, and is then deployed by expanding it to its expanded state. The stent maintains substantially the same length in both its fully compressed and fully expanded states, and in all states between these two states.

The stent according to the present invention can be a self-expanding stent, or a stent that is radially expandable by inflating a balloon or expanded by an expansion member, or a stent that is expanded by the use of radio frequency which provides heat to cause the stent to change its size. The stent may also be coated with coverings of PTFE, dacron, or other biocompatible materials to form a combined stent-graft prosthesis. The vessels in which the stent of the present invention can be deployed include but are not limited to natural body vessels such as ducts, arteries, trachea, veins, ureters and the esophagus, and artificial vessels such as grafts.

Stent Embodiments

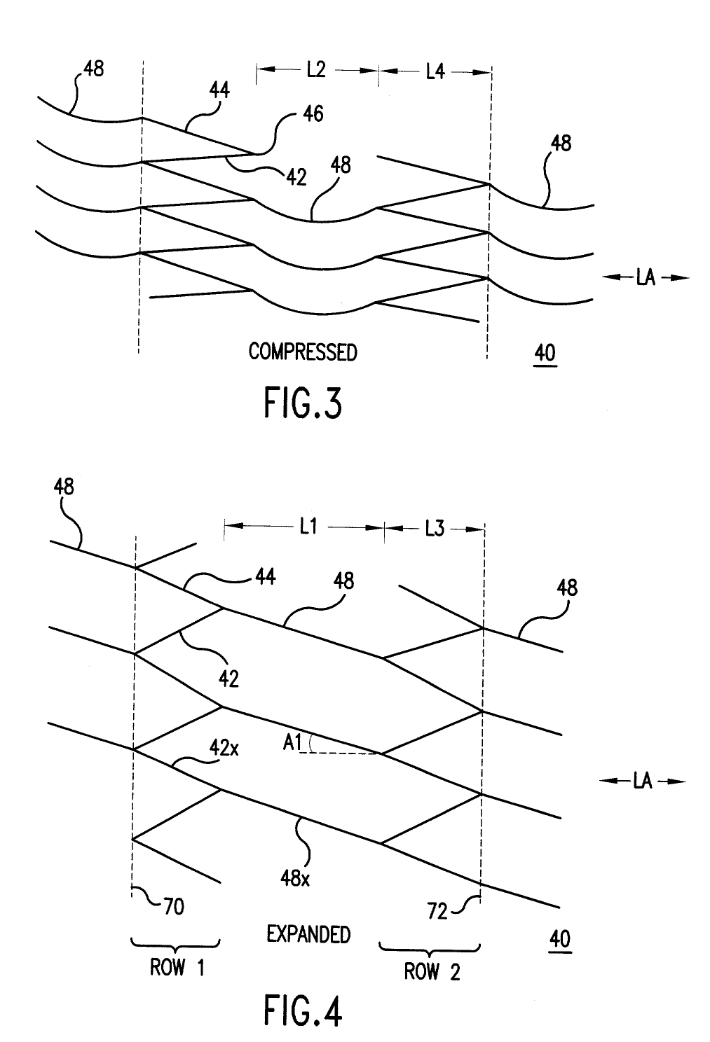

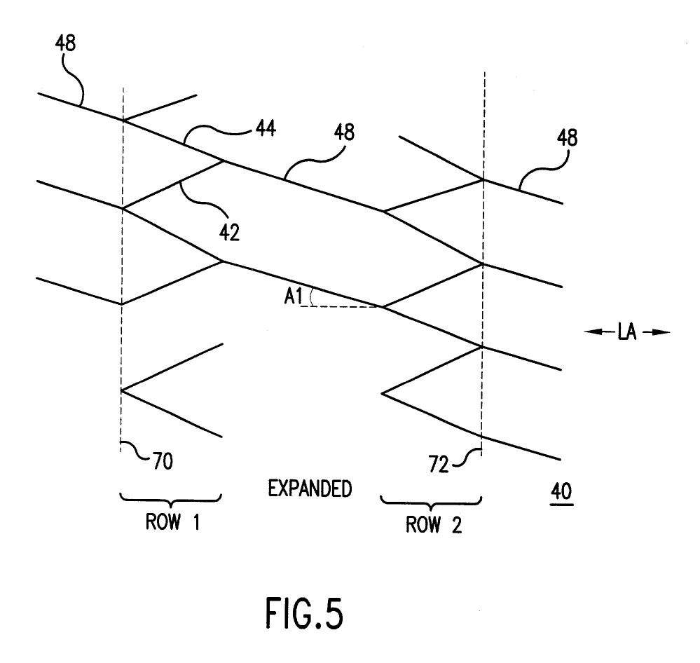

FIGS. 3 and 4 illustrate a portion of a stent 40 according to one embodiment of the present invention. The stent 40 has a tubular configuration and is made up of a plurality of pairs of substantially V-shaped struts connected at their apices, and by connecting one or more connecting members to the apices of selected pairs of V-shaped struts. In particular, the stent 40 has a plurality of pairs of alternating left struts 42 and right struts 44. Each pair of left and right struts 42, 44 is connected at an apex 46 to form a substantially V-shape for the pair. The left strut 42 is defined as being to the left of each apex 46, and the right strut 44 is defined as being to the right of each apex 46. The left struts 42 and right struts 44 are alternating since the left strut 42 of one pair of V-shaped struts is also the right strut of the adjacent pair of V-shaped struts, and the right strut 44 of one pair of V-shaped struts is also the left strut of the adjacent pair of V-shaped struts. In this manner, the alternating left and right struts 42 and 44 extend in an annular manner around the tubular stent 40 to form an annular element. Each apex 46 can be connected to another apex 46 by a connecting member 48. In this embodiment, each connecting member 48 connects adjacent apices 46 along generally the same longitudinal level (see FIG. 3). Therefore, the stent 40 resembles a tubular lattice formed by pairs of V-shaped struts 42, 44 connected to themselves and having their apices 46 connected by the connecting members 48.

The connecting members 48 are generally straight when the stent 40 is in a fully expanded configuration, and are somewhat bowed, curved, arcuate or bent when the stent 40 is in a fully compressed configuration. Each connecting member 48 lies in a generally longitudinal direction along the longitudinal axis LA of the stent 40, but at an angle A1 with respect to the longitudinal axis LA when in the fully expanded configuration (see FIG. 4).

The connecting members 48 are provided to perform two functions. First, the connecting members 48 connect pairs of apices 46. Second, the connecting members 48 function to compensate for the foreshortening experienced by the longitudinal component of each strut 42 and 44, thereby maintaining the stent 40 at substantially the same length at all times. This is accomplished by providing the connecting member 48 with a natural bias and a springy nature, which allows the connecting member 48 to shorten its longitudinal component when compressed. When allowed to expand, the connecting member 48 is biased to return to its natural or original position, which lengthens the longitudinal component of the connecting member 48 to compensate for the foreshortening experienced by the longitudinal component of each strut 42 and 44.

This compensating effect is illustrated in FIGS. 3 and 4. When the stent 40 is in its compressed state, the connecting member 48 has a longitudinal component of L2, which is less than the longitudinal component L1 when the connecting member 48 is in its expanded state. The connecting member 48 is bowed when it is compressed for delivery, but may still be straight when laser-cut. When the stent 40 is in its compressed state, each strut 42, 44 has a longitudinal component of L4, which is greater than the longitudinal component L3 when the struts 42, 44 are in the expanded state. As the stent 40 expands radially with a pre-load, the angle A1 for each connecting member 48 decreases, thereby lengthening the longitudinal component L1 of the connecting member 48 to compensate for the gradual shortening of the longitudinal components L3 of the struts 42, 44. Thus, the difference between L2 and L1 compensates for the difference between L4 and L3 of the struts 42, 44 at both ends of the connecting member 48. The lines 70 and 72 in FIGS. 3 and 4 show that the relevant portion of the stent 40 does not experience any foreshortening and maintains a consistent length through all its states.

In addition, during expansion of the stent 40, it is possible (but not necessary) for the struts 42, 44 in one row (e.g., row 1) to rotate slightly around the longitudinal axis LA of the stent 40 with respect to the struts 42, 44 in an adjacent row (e.g., row 2), so that the struts 42, 44 in one row (e.g., row 1) would now be diagonally offset from the struts 42, 44 in an adjacent row (e.g., row 2).

When the stent 40 is in its fully expanded state, it preferably has an outer diameter that is slightly larger than the inner diameter of the region of the body vessel at which it is to be deployed. This allows the stent 40 to be securely anchored at the desired location and prevents the stent 40 from migrating away from the deployed location.

In the embodiment of FIGS. 3 and 4, all the connecting members 48 are oriented at the same angle and direction throughout the length of the stent 40. Such an orientation would provide the overall structure of the stent 40 with a spiral element at certain intervals, which would enhance the flexibility of the stent 40 at selected locations. In this regard, it is useful for the stent 40 to be provided with varying flexibility or rigidity at different portions or segments along its length to facilitate deployment in body vessels that require such varying flexibility or rigidity, such as curved or angulated body vessels.

The flexibility of the stent 40 can be further varied by omitting one or more connecting members 48 and/or struts 42, 44, such as illustrated in FIG. 5. For example, the connecting member 48x and the strut 42x in FIG. 4 is omitted in FIG. 5, with the stent in FIG. 5 being otherwise identical to the stent in FIG. 4. Omitting connecting members 48 and struts 42, 44 will create “gaps” at one or more locations along the stent 40. These locations can be anywhere along the length and/or the circumference of the stent 40. In addition, varying degrees of flexibility in the stent 40 can be accomplished by varying the patterns of these gaps. A non-limiting example would be to provide a substantially spiral pattern of omitted struts 42, 44 and/or connecting members 48.

A number of materials can be used for both the stent 40 and its struts 42, 44 and connecting members 48, depending on its method of deployment. If used as a self-expanding stent, the stent 40 (including its struts 42, 44 and connecting members 48) is preferably made of a shape memory superelastic metal alloy such as Nitinol, which has the unusual property of “mechanical” memory and trainability. This alloy can be formed into a first predetermined shape above a transition temperature range. The alloy may be plastically deformed into a second shape below the transition temperature range, but the alloy will completely recover to its original (first predetermined) shape when raised back above the transition temperature range. The Nitinol preferably has a composition of about 50% nickel and about 50% titanium. The properties of shape memory alloys such as Nitinol and their use in stents have been well-documented in the literature, and reference can be made to the article by T. W. Duerig, A. R. Pelton and D. Stockel entitled “The Use of Superelasticity in Medicine”, a copy of which is attached hereto and specifically incorporated into this specification by specific reference thereto as though fully set forth herein.

Although the connecting members 48 have been described above as having the same material as the struts 42, 44, it is possible to provide the connecting members 48 with a different material without departing from the spirit and scope of the present invention. Such a material should be springy in nature and should allow the connecting members 48 to be compressed and expanded in the longitudinal direction to compensate for the foreshortening experienced by the struts 42 and 44. Non-limiting examples of such materials can include any of the materials described above for the stent 40.

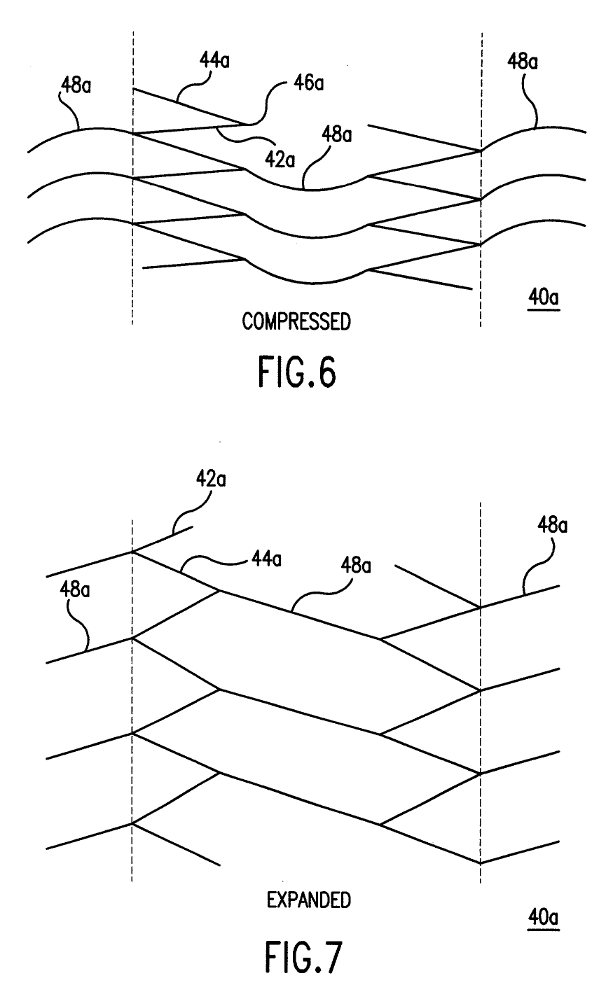

FIGS. 6 and 7 illustrate a portion of a stent 40a according to another embodiment of the present invention. The stent 40a is essentially the same as the stent 40 in FIGS. 3 and 4, except that alternating rows of the connecting members 48a are oriented at opposite angles and directions throughout the length of the stent 40a. Otherwise, the other elements of the stent 40a are the same as the stent 40, and have the same numeral designations except that an “a” has been added.

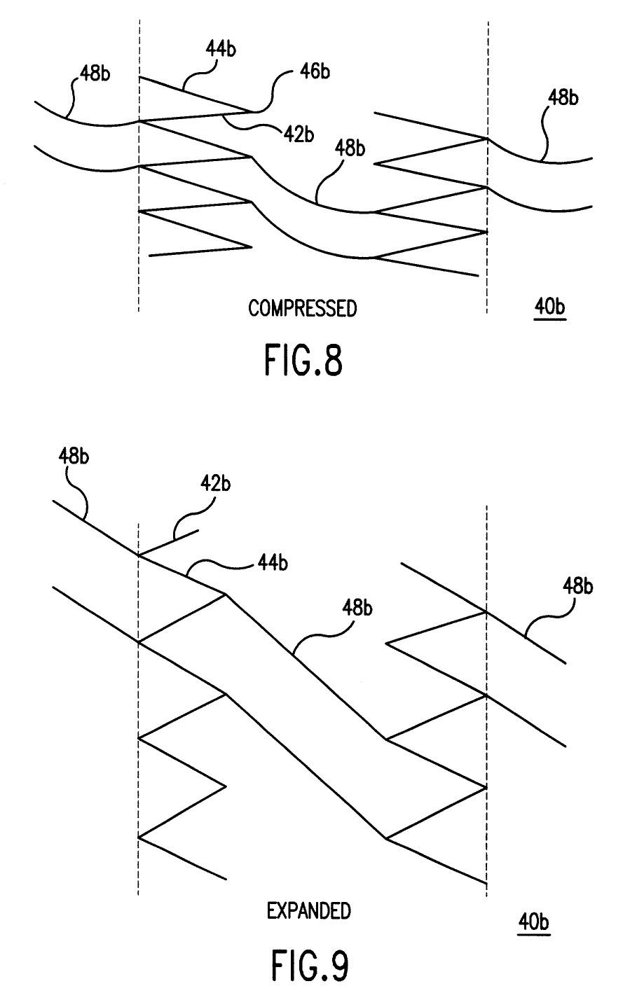

FIGS. 8 and 9 illustrate a portion of a stent 40b according to another embodiment of the present invention. The stent 40b is essentially the same as the stent 40 in FIGS. 3 and 4, except that the connecting members 48b are connected to apices 46b that are diagonally disposed (i.e., at an angle) with respect to each other along the longitudinal axis LA. Otherwise, the other elements of the stent 40b are the same as the stent 40, and have the same numeral designations except that a “b” has been added.

The designs of the stents 40a and 40b provide different types and regions of flexibility (when compared with the stent 40) that may be useful in certain specific applications.

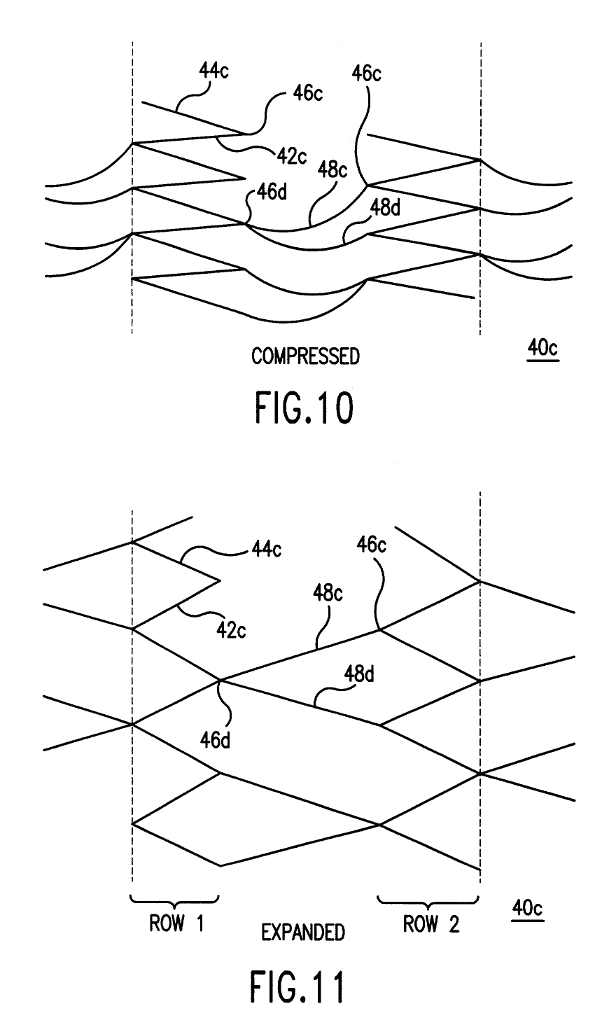

FIGS. 10 and 11 illustrate a portion of a stent 40c according to yet another embodiment of the present invention. The stent 40c is essentially the same as the stent 40 in FIGS. 3 and 4, except that each of two adjacent connecting members 48c and 48d has a first end that is connected to a separate apex 46c in one row (e.g., row 2), and a second end that is connected to a single apex 46d in an adjacent row (e.g., row 1). In addition, when viewed along the same row, each of two circumferentially adjacent apices will be connected to one connecting member, followed by the next circumferentially adjacent apex being connected to two connecting members, and then followed by each of the next two circumferentially adjacent apices being connected to one connecting member, and so on in the same pattern. Otherwise, the other elements of the stent 40c are the same as the stent 40, and have the same numeral designations except that a “c” has been added. Thus, the two connecting members 48c and 48d operate as double-struts, and are effective in providing the portion of the stent 40c with added rigidity.

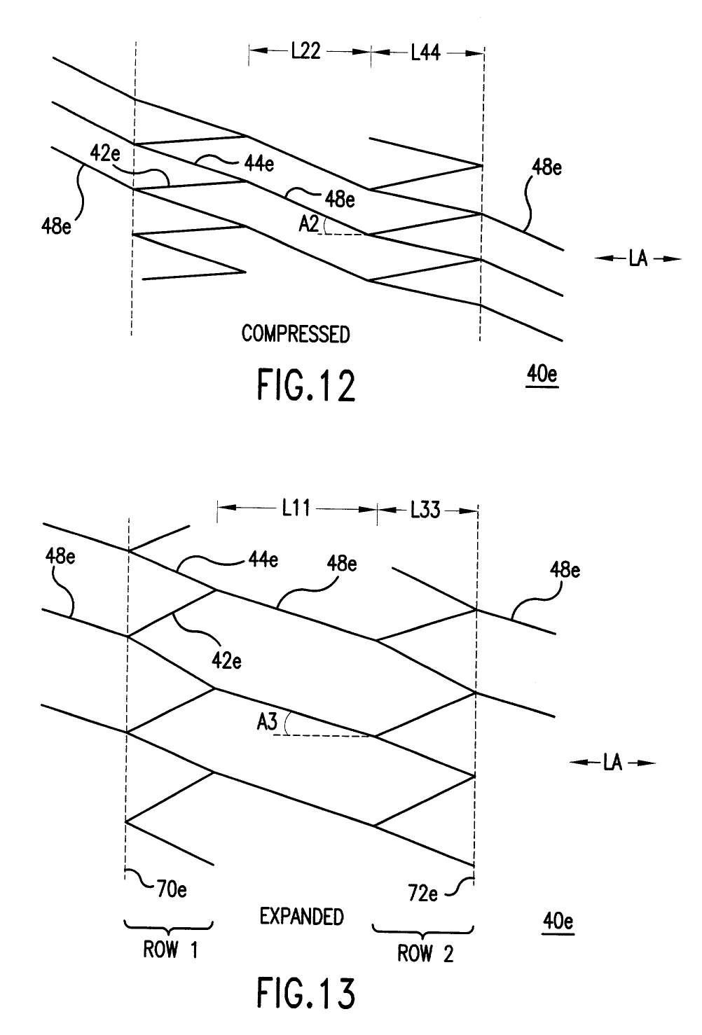

FIGS. 12 and 13 illustrate a portion of a stent 40e according to yet another embodiment of the present invention. The stent 40e is essentially the same as the stent 40 in FIGS. 3 and 4, except that each connecting member 48e is completely straight in both the compressed and the expanded states. Each connecting member 48e lies at an angle A2 with respect to the longitudinal axis LA when in the fully compressed configuration (see FIG. 12), and at an angle A3 with respect to the longitudinal axis LA when in the fully expanded configuration (see FIG. 13), with the angle A2 being greater than the angle A3. The connecting members 48e are also provided with a natural bias and a springy nature, which allows the connecting member 48e to shorten its longitudinal component, and hence increases its angle from A3 to A2, when compressed. When allowed to expand, the connecting member 48e is biased to return to its natural or original position, which lengthens the longitudinal component of the connecting member 48, and hence decreases its angle from A2 to A3, to compensate for the foreshortening experienced by the longitudinal component of each strut 42e and 44e. If the material used for the connecting members 48e is Nitinol, the natural bias or spring nature of the connecting members 48e can be created when the entire stent 40e is being heat-treated to “set” the shape memory of the Nitinol material prior to compression, as explained in greater detail hereinbelow.

This compensating effect is illustrated in FIGS. 12 and 13. When the stent 40e is in its compressed state, the connecting member 48e has a longitudinal component of L22, which is less than the longitudinal component L11 when the connecting member 48e is in its expanded state. The connecting member 48e assumes a greater angle A2 with respect to the longitudinal axis LA when in the compressed state. When the stent 40e is in its compressed state, each strut 42e, 44e has a longitudinal component of L44, which is greater than the longitudinal component L33 when the struts 42e, 44e are in the expanded state. As the stent 40e expands radially, the angle A2 for each connecting member 48e decreases, thereby lengthening the longitudinal component L11 of the connecting member 48e to compensate for the gradual shortening of the longitudinal components L33 of the struts 42e, 44e. Thus, the difference between L22 and L11 compensates for the difference between L44 and L33 of the struts 42e, 44e at both ends of the connecting member 48e. The lines 70e and 72e in FIGS. 12 and 13 show that the relevant portion of the stent 40e does not experience any foreshortening and maintains a consistent length through all its states.

In addition, during expansion of the stent 40e, the struts 42e, 44e in one row (e.g., row 1) would effectively rotate slightly around the longitudinal axis LA of the stent 40e with respect to the struts 42e, 44e in an adjacent row (e.g., row 2), so that the struts 42e, 44e in one row (e.g., row 1) would now be diagonally offset from the struts 42e, 44e in an adjacent row (e.g., row 2). In this manner, the entire length of the stent 40e can experience a helical twist or rotation (when one compares one end of the stent 40e with the opposing end of the stent 40e) when the stent 40e is expanded from the fully compressed configuration to the fully expanded configuration.

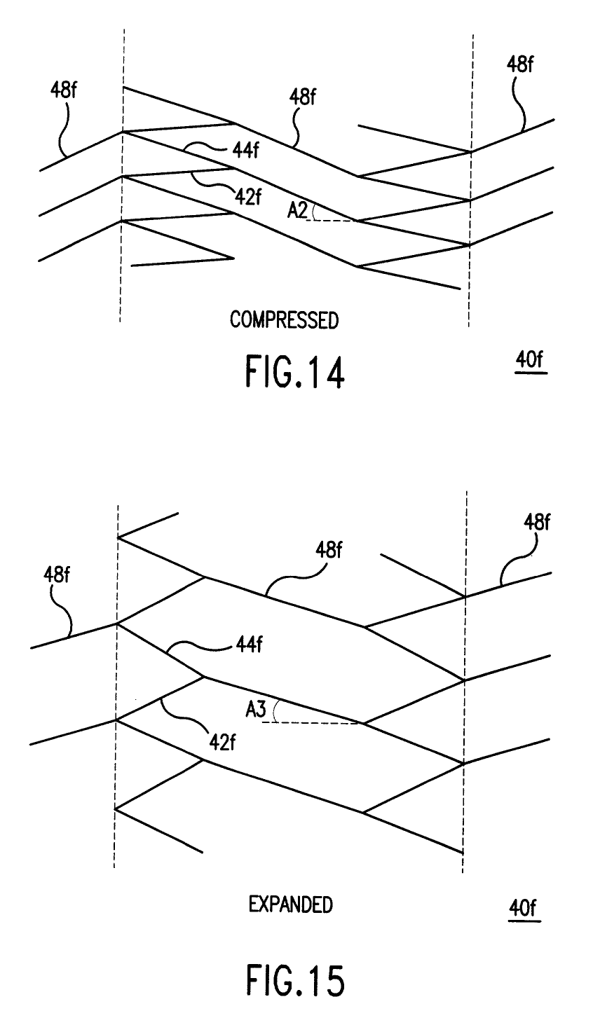

FIGS. 14 and 15 illustrate a portion of a stent 40f according to another embodiment of the present invention. The stent 40f is essentially the same as the stent 40a in FIGS. 6 and 7, except that each connecting member 48f is completely straight in both the compressed and the expanded states. Thus, the stent 40f in FIGS. 14 and 15 combines the principles of the stents 40a and 40e. In other words, alternating rows of the connecting members 48f are oriented at opposite angles and directions throughout the length of the stent 40f, and each connecting member 48f is completely straight in both the compressed and the expanded states. Otherwise, the other elements of the stent 40f are the same as the stent 40a, and have the same numeral designations except that an “f” has been used instead of an “a”.

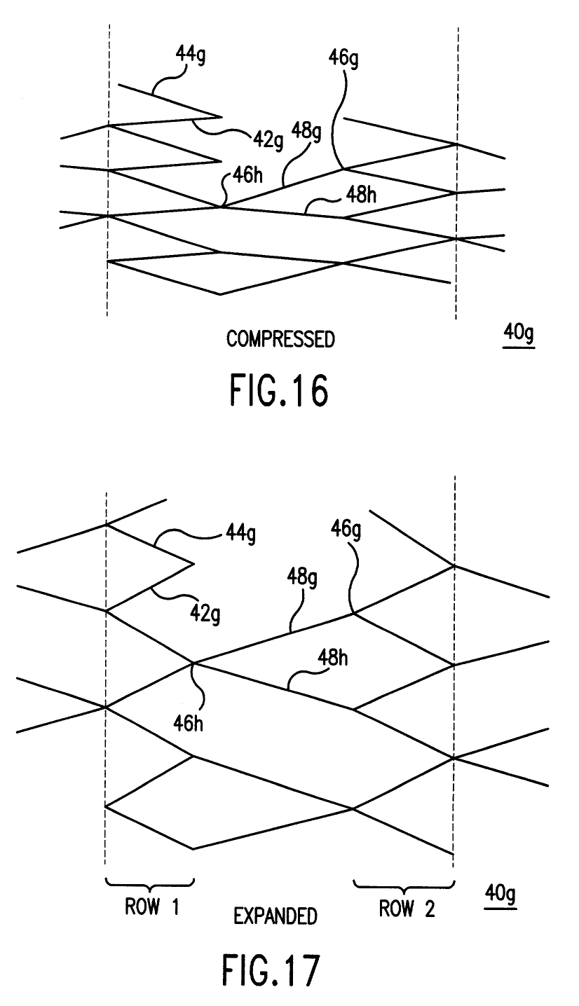

FIGS. 16 and 17 illustrate a portion of a stent 40g according to yet another embodiment of the present invention. The stent 40g is essentially the same as the stent 40c in FIGS. 10 and 11, except that each connecting member 48g is completely straight in both the compressed and the expanded states. Thus, the stent 40g in FIGS. 16 and 17 combines the principles of the stents 40c and 40e. In other words, each of two adjacent connecting members 48g and 48h has a first end that is connected to a separate apex 46g in one row (e.g., row 2), and a second end that is connected to a single apex 46h in an adjacent row (e.g., row 1); and each connecting member 48g, 48h is completely straight in both the compressed and the expanded states. In addition, when viewed along the same row, each of two circumferentially adjacent apices will be connected to one connecting member, followed by the next circumferentially adjacent apex being connected to two connecting members, and then followed by each of the next two circumferentially adjacent apices being connected to one connecting member, and so on in the same pattern. Otherwise, the other elements of the stent 40g are the same as the stent 40c, and have the same numeral designations except that a “g” or “h” has been used instead of a “c” or “d”.

Methods of Manufacture

The stent 40 can be made from one of a number of methods, depending on the material of the stent 40 and the desired nature of deployment. The methods described below apply to the stents 40a-40c and 40e-40g as well.

In a non-limiting first preferred method, the stent 40 is fabricated from a solid Nitinol tube with dimensions that are identical to the stent 40 when it is in the fully compressed state. The pattern of the stent 40 (i.e., its struts 42, 44 and connecting members 48) is programmed into a computer-guided laser cutter or lathe which cuts out the segments between the struts 42, 44 and the connecting members 48 in a manner which closely controls the outside diameter and wall thickness of the stent 40.

After the cutting step, the stent 40 is progressively expanded until it reaches its fully expanded state. The expansion can be performed by an internal expansion fixture, although other expansion apparatus and methods can be used without departing from the spirit and scope of the present invention. The overall length of the stent 40 is preferably consistently maintained throughout the expansion of the stent 40 from its fully compressed to its fully expanded states.

Once the stent 40 has been expanded to its fully expanded state, it is heat-treated to “set” the shape memory of the Nitinol material to the fully expanded dimensions. The stent 40 is then cleaned and electro-polished.

The next step is to compress the stent 40 again into a dimension which allows for delivery into a vessel, either through percutaneous delivery or through minimally invasive surgical procedures. Specifically, the stent 40 must be compressed into a smaller state so that it can be delivered by a delivery device to the desired location of the vessel. Any conventional delivery device could be used, such as but not limited to a tube, catheter, or sheath. The compression is accomplished at low temperatures and involves radial and longitudinal compression to maintain the desired (same) length. This compression is accomplished by cooling the stent 40 to a low temperature, for example, zero degrees Celcius, and while maintaining this temperature, compressing the stent 40 to allow the stent 40 to be inserted inside the delivery device. Once inserted inside the delivery device, the stent 40 is held by the delivery device in the compressed state at room temperature.

While certain methods of manufacture have been described above, it will be appreciated by those skilled in the art that other methods of manufacture can be utilized without departing from the spirit and scope of the present invention.

Deployment Methods

The stent 40 can be deployed by a number of delivery systems and delivery methods. These delivery systems and methods will vary depending on whether the stent 40 is expanded by self-expansion, radial expansion forces, or radio frequency.

While the description above refers to particular embodiments of the present invention, it will be understood that many modifications may be made without departing from the spirit thereof. The accompanying claims are intended to cover such modifications as would fall within the true scope and spirit of the present invention.

What is claimed is:

1. A stent comprising:
a plurality of annular elements, each annular element having a compressed state and an expanded state, wherein each annular element has a longitudinal dimension which is smaller in the expanded state than in the compressed state; and
at least one connecting member connecting adjacent annular elements, the connecting member having a longitudinal dimension which is larger in the expanded state than in the compressed state, and the connecting member being straight when the annular elements are in the compressed state and in the expanded state.

2. The stent of claim 1, wherein each annular element comprises a plurality of alternating struts and apices connected to each other to form a substantially annular configuration.

3. The stent of claim 2, wherein the connecting members are connected to the apices of the adjacent annular members.

4. The stent of claim 2, wherein the plurality of struts comprises left and right struts, with each pair of left and right struts connected to each other at an apex.

5. The stent of claim 2, wherein each strut has a longitudinal dimensional which is smaller when the annular elements are in the expanded state than in the compressed state.

6. The stent of claim 2, wherein each strut has a longitudinal dimensional which is larger when the annular elements are in the compressed state than in the expanded state.

7. The stent of claim 1, wherein the connecting member has a smaller longitudinal dimension when annular elements are in the compressed state than in the expanded state.

8. The stent of claim 1, wherein the stent is made from a shape memory alloy.

9. The stent of claim 8, wherein the shape memory alloy is Nitinol.

10. The stent of claim 1, wherein each connecting member defines an angle with respect to the longitudinal axis of the stent, with the angle being greater when the annular elements in the compressed state than when the annular elements are in the expanded state.

11. The stent of claim 1, wherein the at least one connecting member comprises a plurality of connecting members, with all of the plurality of connecting members oriented at the same angle in the same direction with respect to the longitudinal axis of the stent when the annular elements are in the expanded state.

12. The stent of claim 1, wherein the at least one connecting member comprises a plurality of connecting members that define a plurality of rows of connecting members, wherein the connecting members in one row of connecting members are oriented in a different direction with respect to the connecting members in an adjacent row of connecting members.

13. The stent of claim 1, wherein the connecting member is connected to adjacent annular elements at two separate locations that are along the same longitudinal axis of the stent.

14. The stent of claim 1, wherein the connecting member is connected to adjacent annular elements at two separate locations that are disposed at an angle with respect to the longitudinal axis of the stent.

15. The stent of claim 1, wherein the at least one connecting member comprises a first connecting member and a second connecting member, with the first and second connecting members connected to a first annular element along two separate locations thereof, and with the first and second connecting members connected to an adjacent second annular element at a single location.

16. The stent of claim 15, wherein the two separate locations are two separate apices along the first annular element, and the single location is an apex along the second annular element.

17. The stent of claim 1, wherein each connecting member is devoid of any points of inflection.
